(19) United States
(12) Patent Application Publication
(10) Pub. No.: US 2009/0148546 A1
(43) Pub. Date: Jun. 11, 2009

(54) Methods and compounds fo rthe treatment of obesity and obesity-related disorders

(75) Inventors: Chandan Prasad, New Orleans, LA (US); Julio E. Figueroa, II, New Orleans, LA (US); Parakat Vijayagopal, Kenner, LA (US)

Correspondence Address: ADAMS AND REESE LLP, 4400 ONE HOUSTON CENTER, 1221 MCKINNEY, HOUSTON, TX, 77010, US

(73) Assignee: Board of Supervisors of Louisiana State University and Agricultural and Mechanical College, Baton Rouge, LA (US)

(21) Appl. No.: 12/229,239

(22) Filed: Aug. 21, 2008

Related U.S. Application Data
(63) Continuation of application No. 11/056,765, filed on Feb. 11, 2005, now abandoned.

(51) Int. Cl. A61K 36/87; A61K 31/135; A61K 31/352; A61P 3/04

(52) U.S. Cl. 424/766; 514/627; 514/456

(57) ABSTRACT

In certain embodiments, the invention comprises the use of plant-derived substances to reduce body fat and/or to increase insulin sensitivity in mammals. In certain embodiments, the invention comprises a composition for reducing the amount of fat in the body of a mammal, the composition comprising at least one plant-derived substance which inhibits adipogenesis in the body of the mammal and at least one plant-derived substance which promotes lipolysis in the body of the mammal. In certain embodiments, the invention comprises a composition for reducing the amount of fat in the body of a mammal, the composition comprising at least one plant-derived substance which inhibits adipogenesis in the body of the mammal, at least one plant-derived substance which promotes lipolysis in the body of the mammal, and at least one plant-derived substance which inhibits lipogenesis in the body of the mammal. In certain embodiments, capsaicin may be used in combination with other plant-derived substances to reduce the body fat in a mammalian subject. In one embodiment, an effective amount of a composition comprising capsaicin in combination with grape seed extract and/or genistein and/or caffeine may be administered to a subject to reduce the body fat of the subject. In one embodiment, a composition comprising grape seed extract is administered to a mammalian subject to increase insulin sensitivity.

Embodiments of the present invention relate to the use of plant-derived substances to reduce body fat and/or to increase insulin sensitivity in mammals.

### I. BRIEF DESCRIPTION OF THE DRAWINGS

FIG. 1 shows 3T3-L1 cells cultured in inducing medium followed by maintenance medium for a total of 10 days with no capsaicin. The cells were stained with oil red-O (to stain for intracellular lipid) and photographed.

FIG. 2 shows 3T3-L1 cells cultured in inducing medium followed by maintenance medium for a total of 10 days with 0.5 ug/ml capsaicin. The cells were stained with oil red-O (to stain for intracellular lipid) and photographed.

FIG. 3 shows 3T3-L1 cells cultured in inducing medium followed by maintenance medium for a total of 10 days with 1 ug/ml capsaicin. The cells were stained with oil red-O (to stain for intracellular lipid) and photographed.

FIG. 4 shows the change in body weight (as compared with the control group) for rats consuming differing amounts of combinations of grape seed extract, genistein, capsaicin, and caffeine with a high fat diet. Diet A (g/Kg) comprised 20 g grape seed extract, 600 mg genistein, 600 mg capsaicin, and 12 g caffeine. Diet B (g/Kg) comprised 10 g grape seed extract, 300 mg genistein, 300 mg capsaicin, and 6 g caffeine.

FIG. 5 shows the change in the size (as compared with the control group) of the different fat depots of the rats used in the diet experiment illustrated in FIG. 4.

FIG. 6 shows the results of a lipolysis assay for adipocytes exposed to PBS; 0.3 mM IBMX; 30 mM isoproterenol; 3 mM isoproterenol; 0.3 mM isoproterenol; 500 ug/ml GSE; and ETOH. The data are presented as change in basal glycerol release (an index of lipolysis).

FIG. 7 shows insulin sensitivity indices (ISI) calculated from sera assayed for glucose, free fatty acids (ffa), and insulin as discussed in EXAMPLE 6—mean ISI(gly) and ISI(ffa) are shown.

### II. DESCRIPTION OF THE INVENTION

Obesity is an increase in body weight beyond the limitation of the body's requirements as the result of an excessive accumulation of fat. A person is considered to be overweight if he or she has a body mass index of 25 to 30, and a person is considered to be obese if he or she has a body mass index of greater than 30. Body mass index is determined by dividing a person's weight (in kilograms) by the square of the person's height (in meters). The calculation gives a sense of the amount of fat the person has in his or her body.

The population of the United States is the most obese population in the world. More than 50% of the adult population is overweight, and 20% is considered obese. Being overweight or obese increases the risks for hypertension, coronary heart disease, stroke, diabetes, gallbladder disease, degenerative joint disease, sleep apnea, and certain cancers. Obesity is the second highest cause of preventable death in the United States (exceeded only by cigarette smoking); it is estimated to affect 58,000,000 people and contribute to 300,000 deaths annually. The effects of obesity appear to be most acute in the younger populations (Fontaine, K. R., Redden, D. T., Wang, C., Westfall, A. O., & Allison, D. B., “Years of life lost due to obesity,” JAMA, vol. 28, pp. 187-193 (2003), which is incorporated herein by reference in its entirety). For example, young white men with morbid obesity have a 12-year shorter life span than those with normal body weight (ibid.). Because of the tremendous health implications associated with being overweight or obese, the Centers for Disease Control and Prevention has identified a number of obesity-related conditions as priority areas for “Healthy People 2010,” its comprehensive nationwide health promotion and disease prevention agenda.

The United States government estimates that $100 billion is spent every year to treat obesity. Current treatments include (alone or in combination) diet, exercise, behavior modification, surgical intervention, and pharmacological intervention. While the combination of diet and exercise is the safest treatment, it requires a great deal of discipline and, therefore, has not been very popular. Surgical intervention has been shown to be relatively effective, but it is advised only for morbidly obese people and involves a number of complications. There is a great deal of work being done in the area of pharmacological treatments for obesity.

Obesity is generally classified into two categories based on the site of fat deposition—visceral and nonvisceral (also known as upper-body/android (apple-shaped) and lower-body/gynoid (pear-shaped) obesity, respectively). It is well established that visceral adipose tissue is associated with greater morbidity and mortality, particularly hypertension, hyperlipidemia, and insulin resistance (Pi-Sunyer, F. X., “Comorbidities of overweight and obesity: current evidence and research issues,” Medicine & Science in Sports & Exercise, vol. 31 (11 Suppl.), pp. S602-8 (1999), which is incorporated herein by reference in its entirety). Data show that weight loss generated by diet, exercise, or pharmacotherapy decreases visceral adipose tissue and improves hypertension, hyperlipidemia, and insulin resistance (Bray, G. A. & Tartaglia, L. A., “Medicinal strategies in the treatment of obesity,” Nature, vol. 404, pp. 672-677 (2000), which is incorporated herein by reference in its entirety).

There are currently a number of strategies for developing drugs to treat obesity, including (1) reducing food intake either by amplifying inhibitory effects of anorexigenic signals or factors (those that suppress food intake) or by blocking orexigenic signals or factors (those that stimulate food intake); (2) blocking nutrient absorption (especially fat) in the gut; (3) increasing thermogenesis (the physiologic process of heat production in the body) by uncoupling fuel metabolism from the generation of adenosine triphosphate (ATP) and thereby dissipating food energy as heat; (4) modulating fat metabolism or storage by regulating fat synthesis (lipogenesis), fat breakdown (lipolysis), adipose differentiation (adipogenesis), and/or fat cell death (apoptosis); and (5) modulating the central controller regulating body weight by altering the internal reference value sought by the controller, or by modulating the primary afferent signals regarding fat stores that are analyzed by the controller (this approach would have the potential advantage of forcing the endogenous controller to regulate multiple pathways of energy balance and minimize compensation (Bray, G. A. & Tartaglia, L. A., “Medicinal strategies in the treatment of obesity,” Nature, vol. 404, pp. 672-677 (2000), which is incorporated herein by reference in its entirety).

In many respects, obesity is analogous to hypertension, a chronic disease modulated by several levels of feedback regulation. Several generations of anti-hypertensive drugs have shaped our understanding of the blood pressure feedback system, and a similar situation is predicted for anti-obesity medications. Like hypertension, it is reasonable to expect that for many obese patients, effective therapy will involve chronic use of more than one drug. However, experiences with many drugs aimed at reducing food intake have not been very successful due to serious side effects. Thus, any approved drug will be required to meet high standards of safety.

Anorectic centrally acting agents such as fenfluoramine, phentermine, dexfenfluramine and sibutramine have been successful in treating obesity; however, some of these agents have been removed from the market due to serious side effects (Bray, G. A. & Tartaglia, L. A., “Medicinal strategies in the treatment of obesity,” Nature, vol. 404, pp. 672-677 (2000), which is incorporated herein by reference in its entirety). Orlistat, an intestinal lipase inhibitor, has been shown to cause weight loss in subjects who tolerate a low fat diet, but the drug causes gastrointestinal side effects (ibid.). Currently, there is no available pharmacotherapy that facilitates a decrease in fat storage.

At any given time, adipose tissue mass reflects the number and average volume of adipocytes (animal connective tissue cells specialized for the synthesis and storage of fat), both of these parameters being under complex control. The major contributor to adipocyte volume is cytoplasmic triglyceride, which is determined by the balance between lipogenesis (the production of fat) and lipolysis (the breakdown of fat). Both lipogenesis and lipolysis have a number of positive and negative regulators (see TABLE 1 below). Adipocyte number is determined by the relative rate of preadipocyte replication and their differentiation into adipocytes. Preadipocytes are precursor cells from which adipocytes are derived. Preadipocytes multiply. They also differentiate into mature adipocytes after undergoing signaling that appears to involve certain nuclear binding proteins (e.g., peroxisome proliferator activated-receptor (PPAR) gene productions). Mature adipocytes are terminally differentiated and, therefore, do not multiply (see Van, R. L. & Roncari, D. A., “Complete differentiation of adipocyte precursors. A culture system for studying the cellular nature of adipose tissue,” Cell & Tissue Res., vol. 195, pp. 317-29 (1978), which is incorporated herein by reference in its entirety). Adipocyte number is also determined by the rates of cell loss by apoptosis (the programmed destruction of cells from within, see Prins, J. B, Walker, N. I., Winterford, C. M. & Cameron, D. P., “Human adipocyte apoptosis occurs in malignancy,” Biochem. Biophys. Res. Commun., vol. 205, pp. 625-30 (1994), which is incorporated herein by reference in its entirety) and dedifferentiation. Dedifferentiation is a novel concept. Generally, it involves the theory that high expression of certain molecules (for instance, leptin) can cause the return of differentiated adipocyte cells into preadipcytes, which leads to a loss of mature adipocytes and, therefore, fat (see Zhou, Y. T., Wang, Z. W., Higa, M., Newgard, C. B. & Unger, R. H., “Reversing adipocyte differentiation: implications for treatment of obesity,” Proc. Nat. Acad. Sci. of the U.S.A., vol. 96, pp. 2391-5 (1999)), which is incorporated herein by reference in its entirety).

TABLE 1

| Modulators | Increase | Decrease |
|---|---|---|
| Fat Cell Volume | | |
| 1. Lipogenesis | Insulin, EGF, | GH, TNF-∝ |
| | Angiotensin II, | |
| | glucocorticoids | |
| 2. Lipolysis | GIP, GH, Leptin, TNF- | Insulin, NPY |
| | ∝, NPY-antagonist | |
| Fat Cell Number | | |
| 1. Differentiation of | Insulin, glucocorticoids | EGF, TNF-∝ |
| preadipocytes to adipocytes | | |
| 2. Dedifferentiation of | Leptin | |
| adipocytes to preadipocytes | | |
| 3. Apoptosis of: | | |
| (a) adipocytes | Leptin, TNF-∝, NPY- | NFκB, bcl-2, |
| | antagonist | Insulin |
| (b) preadipocytes | Retinoic acid, leptin, | |
| | TNF-∝ | |

(see Cowherd, R. M., Lyle, R. E. & McGehee, R. E., Jr., “Molecular regulation of adipocyte differentiation,” Seminars in Cell & Dev. Biol., vol. 10, pp. 3-10 (1999); Langin, D., Lucas, S. & Lafontan, M., “Millennium fat-cell lipolysis reveals unsuspected novel tracks,” Hormone & Metab. Res., vol. 32, pp. 443-52 (2000); Rangwala, S. M. & Lazar, M. A., “Transcriptional control of adipogenesis,” Ann. Rev. Nutrition., vol. 20, pp. 535-59 (2000); Rosen, E. D., Walkey, C. J., Puigserver, P. & Spiegelman, B. M., “Transcriptional regulation of adipogenesis. Genes & Development,” vol. 14, pp. 1293-1307 (2000); Sethi, J. K. & Hotamisligil, G. S., “The role of TNF alpha in adipocyte metabolism,” Seminars in Cell & Dev. Biol., vol. 10, pp. 19-29 (1999); Sorisky A., Magun R. & Gagnon, A. M., “Adipose cell apoptosis: death in the energy depot,” Int. J. Obesity & Related Metab. Disorders, vol. 4 (24 Suppl.), pp. S3-7 (2000); Spiegelman, B. M., Puigserver, P. & Wu, Z., “Regulation of adipogenesis and energy balance by PPARgamma and PGC-1,” Int. J. Obesity & Rel. Metab. Dis., vol. 4 (24 Suppl.), pp. S8-10 (2000); Vernon, R. G., Barber, M. C. & Travers, M. T. “Present and future studies on lipogenesis in animals and human subjects,” Proc. Nutrition Soc., vol. 58, pp. 541-9 (1999), each of which is incorporated herein by reference in its entirety).

A change in adipose tissue mass must involve a change in adipocyte number, adipocyte volume, or both. It has been shown that a reduction in adipocyte volume and number occurs with the loss of adipose tissue (Mauriege, P., Imbeault, P., Langin, D., Lacaille, M., Almeras, N., Tremblay, A. & Despres, J. P., “Regional and gender variations in adipose tissue lipolysis in response to weight loss,” J. Lipid Res., vol. 40, pp. 1559-71 (1999), which is incorporated herein by reference in its entirety). Current evidence suggests that a significant increase or decrease in fat mass involves a change in both parameters. It has also been observed that during weight gain, adipocyte volume increases to a “critical” point, after which recruitment of new cells occurs (Bjorntorp, P., “Adipose tissue distribution and function,” Int. Journal Obesity, vol. 2 (15 Suppl.), pp. 67-81 (1991), which is incorporated herein by reference in its entirety).

An effective strategy for controlling obesity will involve the development of substances that modulate one or more aspects of fat metabolism and storage, resulting in diminution of the fat depot. For many reasons, substances that deplete the fat depot are more desirable than substances directed to curb appetite or desire to eat. Substances known to decrease food intake or desire to eat are generally known to act at the level of the central nervous system and involve one or more of the ever present neurotransmitters (e.g., norepinephrine, serotonin, and dopamine). These neurotransmitters are ubiquitously distributed throughout the central nervous system. They are associated with a variety of other functions in addition to food intake, and modulators of these neurotransmitters are not exclusively specific to food intake signals. The side effects associated with these neurotransmitters stem from these other effects on central function. Therefore, substances that act at the level of the central nervous system are prone to have serious side effects (Carek, P. J. & Dickerson, L. M., “Current concepts in the pharmacological management of obesity,” Drugs, vol. 57, pp. 883-904 (1999), which is incorporated herein by reference in its entirety).

Substances capable of affecting fat metabolism and storage will be more likely to act at peripheral sites. Adipocyte modulation occurs primarily through signals other than neurotransmitters, although many of these factors also have a multiplicity of effects on a variety of cells. In general, the more specific the cell spectrum and effect, the less likely there are to be untoward side effects.

Reduction in the fat depot, particularly visceral fat, has many beneficial medical consequences. It is now well established that in determining the risk of an individual developing certain metabolic sequelae, the distribution of body fat is of greater importance than the degree of excess adipose tissue per se. Upper body or visceral obesity is closely associated with cardiovascular disease, diabetes type II, and syndrome X (Anonymous, “Overweight, Obesity, and Health Risk,” Archives of Internal Medicine, vol. 160, pp. 898-904 (2000), which is incorporated herein by reference in its entirety). In contrast, individuals with comparable amounts of adipose tissue stored in the femoral or gluteal depot (lower body obesity) have much lower risk for morbidity and mortality from the above-mentioned metabolic syndromes. It has been shown that the selective reduction in visceral adiposity with diet and exercise is accompanied by improvements in intermediary metabolism (Fujioka S., Matsuzawa Y., Tokunaga K., Kawamoto T., Kobatake T., Keno Y., Kotani K., Yoshida S. & Tarui S., “Improvement of glucose and lipid metabolism associated with selective reduction of intraabdominal visceral fat in premenopausal women with visceral fat obesity,” Int. Journal Obesity, vol. 15, pp. 853-9 (1991), which is incorporated herein by reference in its entirety).

A number of studies have described the use of plant-derived products for the treatment of obesity, including plant-derived caffeine (guarana or gotu kola), phytoestrogens (genistein), and capsaicin.

Caffeine in combination with ephedra alkaloids is one of the most widely used supplements for weight loss. Several studies have reported achieving significant weight loss with this combination (Herber, D., “Herbal preparations for obesity: are they useful?”, Primary Care, Clinics in Office Practice, Vol. 30 (2), 2003; Boozer, C. N., Daley, P. A., Homel, P., Solomon, J. L., Blanchard, D., Nasser, J. A., Strauss, R. & Meredith, T., “Herbal ephedra/caffeine for weight loss: a 6-month randomized safety and efficacy study; Int. J. Bes. Relat. Metab. Disord. vol. 26(5), pp. 593-604 (2002); Molnar, D., Torok, K., Erhardt, E., & Jeges S., “Safety and efficacy of treatment with an ephedrine/caffeine mixture. The first double-blind pilot study in adolescents”, Int. J. Obes. Relat. Metab. Disord. vol. 24(12), pp. 1573-8 (2000), each of which is incorporated herein by reference in its entirety). Patients who achieve only a small amount of weight loss during dietary therapy, and have a tendency to regain weight, are characterized by lower energy expenditure, lower sympathetic activity, and a reduced ability to mobilize fat stores, as compared with patients who are more successful at losing and keeping off weight. This suggests that a low-energy-output phenotype is at high risk for weight gain and obesity, irrespective of whether this is due to a low resting metabolic rate and/or physical inactivity. The low-energy-output phenotype is associated with impaired appetite control, which is improved if energy output is increased. This is the basis for pharmacological stimulation of energy expenditure as a tool to improve the outcome of obesity treatment. Agents that stimulate adrenergic neurons are particularly suitable because they offer mechanisms for inhibiting hunger and for stimulating energy expenditure, lipolysis, and fat oxidation. Targets are the leptin receptors, the sympathetic nervous system and its peripheral beta-adrenoceptors, selective thyroid hormone derivatives, and stimulation of the mitochondrial uncoupling proteins. The Ephedrine/caffeine combination, for example, possesses thermogenic properties due to activation of the sympathoadrenal system and thus increases energy expenditure (Astrup, A. (2000) Thermogenic drugs as a strategy for treatment of obesity. Endocrine. October; 13(2):207-12, which is incorporated herein in its entirety). However, this treatment is also accompanied by serious side effects such as hypertension, palpitation, tachycardia, stroke, and seizures (Herber, D., “Herbal preparations for obesity: are they useful?” Primary Care, Clinics in Office Practice, Vol. 30 (2), 2003)).

The efficacy of caffeine alone in causing weight loss has also been investigated. Caffeine activates the sympathetic nervous system (see Chen, M. D., Lin, W. H., Song, Y. M., Lin, P. Y., & Ho, L. T., “effect of caffeine on the level of brain serotonin and catecholamine in the genetically obese mice”, Zhonghua Yi Xue Za Zhi (Taipei), vol. 53, pp 257-61 (1994), which is hereby incorporated by reference herein in its entirety), which can lead to weight loss—as noted, activation of the sympathetic system causes thermogenesis which results in loss of energy as heat (Macdonald, I. A., “Advances in our understanding of the role of the sympathetic nervous system in obesity”, Int. J. Obes. Relat. Metab. Disord. vol. 19 (suppl. 7), pp. S2-7 (1995), which is hereby incorporated by reference herein in its entirety). Caffeine has been shown to decrease body fat in genetically obese mice (0b/0b) (see Chen, M. D., Lin, W. H., Song, Y. M., Lin, P. Y., & Ho, L. T., “effect of caffeine on the level of brain serotonin and catecholamine in the genetically obese mice”, Zhonghua Yi Xue Za Zhi (Taipei), vol. 53, pp 257-61 (1994), which is hereby incorporated by reference herein in its entirety). Caffeine has also been shown to increase oxygen consumption, fat oxidation and serum free fatty acids in normal and obese subjects (Acheson, K. J., Zahorska-Markiewicz, B., Pittet, P. et al., “Caffeine and coffee: their influence on metabolic rate and substrate utilization in normal weight and obese individuals”, Am. J. Clin. Nutr., vol. 33, pp. 989-997 (1980), which is hereby incorporated by reference herein in its entirety). Caffeine stimulates thermogenesis. Oxygen consumption is an index of metabolic rate. Higher oxygen consumption coupled with increased thermogenesis indicates loss of energy as heat. Increased fat oxidation indicates accelerated loss of fat depot. It is believed, therefore, that caffeine-mediated increases in oxygen consumption and fat oxidation can lead to weight loss in humans (ibid.).

In other studies, caffeine has been shown to increase lipolysis (Jung, R. T., Shetty, P. S., James, W. P., Barrand, M. A., & Callingham, B. A., “Caffeine: its effect on catecholamines and metabolism in lean and obese humans”, Clin. Sci. (Lond), vol. 60(5), pp. 527-35 (1981), which is hereby incorporated by reference herein in its entirety).

Some studies have found elevation of blood pressure with caffeine administration (Noble, R. A., “A controlled clinical trial of the cardiovascular and psychological effects of phenylpropanolamine and caffeine”, Drug Intell. Clin. Pharm., vol. 22, pp. 296-9 (1988), which is hereby incorporated by reference herein in its entirety.).

Capsicum fruits or red peppers are widely used by humans as spices. The active ingredient of hot peppers is capsaicin. It was first isolated by Thresh more than a century ago (Thresh, L. T., “Isolation of capsaicin”, Pharm. J., vol. 6, pp. 941 (1846), which is hereby incorporated by reference herein in its entirety). Studies involving animals have reported that capsaicin increases body temperature. Addition of chili pepper to meals in humans has been shown to accelerate dissipation of surplus calories by sympathetically mediated thermogenesis as indicated by elevated oxygen consumption. This loss of calories can lead to weight loss (see Henry, C. J. K. & Emery B., Effect of spiced food on metabolic rate. Human Nutrition: Clin Nutr. 1986, 40C:165-168; Dib, B., “Effects of intrathecal capsaicin on autonomic and behavioral heat loss responses in the rat”, Pharmacol. Biochem. Behav., vol. 28, pp. 65-70 (1987), each of which is hereby incorporated by reference herein in its entirety). Studies involving animals have also reported that capsaicin (1) stimulates catecholamine secretion (catecholamines activate the sympathetic nervous system which promotes thermogenesis and loss of calories as heat (Watanabe, T., Kawada, T., Kato, T., Harada, T., & Iwai, K., “Effects of capsaicin analogs on adrenal catecholamine secretion in rats,” Life Sci., Vol. 54, pp. 369-374 (1994), which is hereby incorporated by reference herein in its entirety)); (2) enhances energy expenditure (Kawada, T., Sakebe, S., Aoki, N., Watenabe, T., Higeta, K., Iwai, K. & Suigimoto, E., “Intake of sweeteners and pungent ingredients increases the thermogenin content in brown adipose tissue of rat, J. Agric. Food Chem., vol. 39, pp. 651-54 (1991), which is hereby incorporated by reference herein in its entirety); and (3) suppresses body fat accumulation (thermogenin is a protein that helps generate heat in a cell by allowing protons to go back into the mitochondrion without having to go through ATP synthase; it is found in brown adipose tissue, which is rich in mitochondria; hibernating animals use thermogenin to maintain their temperature while keeping their metabolic rate at a minimum; an increase in the level of thermogenin is indicative of increased calorie loss through thermogenesis and heat loss) (Kawada, T., Hagihara, K., & Iwai, K., “Effects of capsaicin on lipid metabolism in rats fed a high fat diet”, J. Nutr., vol. 116, pp. 1272-78 (1986), which is hereby incorporated by reference herein in its entirety). Capsaicin has also been shown to stimulate fat oxidation and thermogenesis in humans (Henry, C. J. & Emery, B., “Effect of spiced food on metabolic rate”, Hum. Nutr Clin. Nutr., vol. 40, pp. 165-8 (1986); Yoshioka, M., St-pierre, S., Suzuki, M. et al., “Effects of red pepper added to high-fat and high-carbohydrate meals on energy metabolism and substrate utilization in Japanese women”, Br. J. Nutr., vol. 80, pp. 503-10 (1998), each of which is hereby incorporated by reference herein in its entirety).

Capsaicin interacts with a family of receptors collectively known as vanilloid receptors to elicit its biologic activities (see Szallasi, A. & Blumberg, P. M., “Vanilloid (capsaicin) receptors and mechanisms, Pharmacol. Rev. vol. 51, pp. 159-212 (1999), which is hereby incorporated by reference herein in its entirety). Because of its pungency, however, capsaicin has limited usefulness to treat obesity in humans.

Capsicum sp. (Red Pepper) contains many analogs of capsaicin, and the analogs containing C14 to C20 alkyl side chains exhibit no pungency (Ohnuki, K., Niwa, S., Maeda, S., Inoue, N., Yazawa, S., & Fushiki, N., “CH-19 sweet, a non-pungent cultivar of red pepper, increases body temperature and oxygen consumption in humans”, Biosci. Biotechnol. Biochem., vol. 65, pp. 2033-36 (2001), which is hereby incorporated by reference herein in its entirety). As used herein, the term “analog” means a substance that is structurally similar to an original molecule but differs in composition and which may or may not have some or all of the activities of the original molecule. For example, capsaicin has many of its effects through its binding with a variety of vanilloid receptors. A similar molecule, capsazepine, has inhibitory effects on capsaicin action by competing with its receptors. On the other hand, olvanil, another similar molecule, has some but not all of the effects of capsaicin because it has a different receptor binding pattern (see Szallasi, A., Di Marzo, V., New perspectives on enigmatic vanilloid receptors. Trends Neurosci. 2000 October, 23(10):491-7, which is hereby incorporated by reference herein in its entirety). Some of these nonpungent analogs are known to stimulate fat metabolism (Kim, K. M., Kawada, T., Ishihara, K., Inoue, K., & Fushiki, T., “Increase in swimming endurance capacity of mice by capsaicin-induced adrenal catecholamine secretion, Biosci. Biotechnol. Biochem., vol. 61, pp. 1718-23 (1989), which is hereby incorporated by reference herein in its entirety). However, these analogs are minor components of Capsicum sp. which are very difficult to isolate.

Analogs of Caffeine include without limitation: 3,7-Dimethyl-1-propargylxanthine and others containing ethyl, propyl, allyl, propargyl and other substituents in place of methyl at 1-, 3- and 7-positions of caffeine.

CH-19 Sweet, a nonpungent pepper, is a rich source of two capsaicin analogs named capsiate and dihydrocapsiate (Kobata, K., Todo, T., Yazawa, S., Iwai, K., & Watanabe, T., “Novel capsacinoid-like substances, capsiate and dihydrocapsiate from the fruits of a non-pungent cultivar, CH-19 sweet, of pepper (Capsicum annuum L.)”, J. Agric. Food Chem., vol. 46, pp. 1695-97 (1998), which is hereby incorporated by reference herein in its entirety). Although capsiate and capsaicin have identical acyl residues, they differ in the aromatic portion—capsaicin has vanillylamine and capsiate has vanillylalcohol (Masuda, Y., Haramizu, S., Oki, K., Ohnuki, K., Watanabe, T., Yazava, S., Kazada, T., Hashizume, S., & Fushiki, T., “Upregulation of uncoupling proteins by oral administration of capsiate, a nonpungent capsaicin analog”, J. Appl. Physiol., vol. 95, pp. 2408-15 (2003), which is hereby incorporated by reference herein in its entirety). Studies have shown that capsiate prevents body fat accumulation in mice to the same extent as capsaicin (Ohnuki, K., Haramizu, S., Oki, K., Watanabe, T., Yazava, S., & Fushiki, T., “Administration of capsiate, a nonpungent capsaicin analog, promotes energy metabolism and suppresses body fat accumulation in mice,” Biosci. Biotechnol. Biochem., vol. 65, pp. 2735-40 (2001), which is hereby incorporated by reference herein in its entirety). Capsiate has also been reported to increase thermogenesis and energy consumption in humans (Ohnuki, K., Niwa, S., Maeda, S., Inoue, N., Yazawa, S., & Fushiki, N., “CH-19 sweet, a non-pungent cultivar of red pepper, increases body temperature and oxygen consumption in humans”, Biosci. Biotechnol. Biochem., vol. 65, pp. 2033-36 (2001), which is hereby incorporated by reference herein in its entirety).

There are other capsaicin analogs, including Capsaicin (trans-8-methyl-N-vanillyl-6-nonenamide), Civamide, Dihydrocapsaicin (8-methyl-N-vanillyl-nonamide), Homocapsaicin (trans-9-methyl-N-vanillyl-7-decenamide), Homodihydrocapsaicin (9-methyl-N-vanillyl-decamide), NE-19550 (Olvanil), NE-21610, NE-28345 (N-oleyl-homovanillamide), Nonivamide, Nordihydrocapsaicin (7-methyl-N-vanillyl-octamide), and Resiniferatoxin. There may also be molecules which are structurally dissimilar to capsaicin but similar in function. We will refer to such molecules as “capsaicinoids.”

Genistein is one of the major phytoestrogens from the isoflavone class that interacts with estrogen receptor (Bolego, C., Poli, A., & Paoletti, R., “Phytoestrogens: pharmacological and therapeutic perspectives, Curr. Drug Targets, vol. 4, pp. 77-87 (2003), which is hereby incorporated by reference herein in its entirety). Soybeans and kudzu are very rich sources of genistein. Other sources include clovers, alfalfa, lentils, oat, barley, rye, wheat, and corn. A number of studies have examined the effect of soy protein on obesity in humans and animals (see Bosello, O., Cominacini, L., Zocca, I., et al., “Short- and long-term effects of hypocaloric diets containing proteins of different sources on plasma lipids and apoproteins of obeses subjects, Ann. Nutr. Metab., vol. 32, pp. 206-14 (1998); Yamashita, T., Sasahara, T., Pomeroy, S. E., Collier, G., & Nestel, P. J., “Arterial compliance, blood pressure, and plasma lipids in women are improved with weight reduction equally with meat-based diet and plant-based diet, Metabolism, vol. 47, pp. 1308-14 (1998); Saito, M., “Effect of soy peptides on energy metabolism in obese animals, Nutr. Sci. Soy Proteins, vol. 12, pp. 91-4 (1991); Aoyama, T., Fukui, K., Takamatsu, K., et al., “Soy protein isolate and its hydrolysate reduce body fat of dietary obese rats and genetically obese mice (yello kk)”, Nutrition, vol. 16, pp. 349-54 (2000), each of which is hereby incorporated by reference herein in its entirety). However, very few studies have investigated the direct effects of genistein on obesity.

In healthy, postmenopausal women, genistein consumption was associated with weight loss and reduced body mass index (Goodman-Gruen, D. & Kritz-Silverstein, D., “Usual dietary isoflavone intake and body fat composition in postmenopausal women, Menopause, vol. 10, pp 427-32 (2003) which is hereby incorporated by reference herein in its entirety), as well as reduced risk for cardiovascular disease (Goodman-Gruen, D. & Kritz-Silverstein, D., “Usual dietary isoflavone intake is associated with cardiovascular disease risk factors in postmenopausal women”, J. Nutr., vol. 131, pp 1206-6 (2001), which is hereby incorporated by reference herein in its entirety). In rat adipocytes, acute administration of genistein was shown to decrease both basal and insulin-stimulated lipogenesis, and to stimulate lipolysis (Szkudelska, K., Nagowski, L., & Szkudelski, T., “Genistein affects lipogenesis and lipolysis in isolated rat adipocytes, J. Steroid Biochem, Mol. Biol., vol. 75, pp. 265-71 (2000), which is hereby incorporated by reference herein in its entirety). Genistein has also been shown to inhibit adipogenesis and stimulate lipolysis in 3T3-L1 adipocytes (Harmon, A. W. & Harp, J. B., “Differential effects of flavanoids on 3T3-L1 adipogenesis and lipolysis, Am. J. Physiol. Cell. Physiol., vol. 280, pp. C807-13 (2001), which is hereby incorporated by reference herein in its entirety). And long term administration of low doses of genistein has been shown to inhibit lipogenesis in C57/BL6 mice (Naaz, A., Yellayi, S., Zakroczymski, M. A., Bunick, D., Doerge, D. R., Lubahn, D. B., Helferich, W. G., & Cooke, P. S., “The soy isoflavone genistein decreases adipose deposition in mice’, Endocrinology, vol. 144, pp. 3315-20 (2003), which is hereby incorporated by reference herein in its entirety).

Genistein analogs include without limitation: Orobol (a genistein analog with an additional hydroxy group at the 3′ position), Genistin (genistein glycosides with a glucose moiety at 7 position), sophoricoside (genistein glycosides with a glucose moiety at 4′ position), and benzimidazolone analogs (NS004 and NS1619) of genistein.

Grape Seed Extract (GSE) is a rich source of proanthocyanidins. Proanthocyanidins are naturally occurring plant metabolites common to fruits, vegetables, nuts, seeds, flowers, and bark. Plant-based sources of proanthocyanidins include wine, cranberries, and the leaves of bilberry, birch, ginkgo, and hawthorn (Cos P., Hermans N., Calomme M., Maes L., de Bruyne T., Pieters L., Vlietinck A. J., vanden Berghe D., “Comparative study of eight well-known polyphenolic antioxidants,” Journal of pharmacy and pharmacology, 55:(2003), p. 1291-1297, which is incorporated herein by reference in its entirety). Also known as procyanidins, these substances are the primary precursors of the blue, violet, and red pigments in plants. These compounds are part of a specific group of polyphenolic compounds known as the flavonoids. Proanthocyanidins are high-molecular-weight polymers comprised of the monomeric unit flavan-3-ol ((+) catechin and (−) epicatechin).

The biological properties of flavonoids, including proanthocyanidins, have been extensively reviewed (Cos P., Hermans N., Calomme M., Maes L., de Bruyne T., Pieters L., Vlietinck A. J., vanden Berghe D., “Comparative study of eight well-known polyphenolic antioxidants,” Journal of pharmacy and pharmacology, 55:(2003), p. 1291-1297, which is incorporated herein by reference in its entirety). The free radical scavenging abilities of proanthocyanidins have been well documented. In vivo studies have shown grape seed proanthocyanidin extract is a better free radical scavenger and inhibitor of oxidative tissue damage than vitamin C, vitamin E succinate, vitamin C and vitamin E succinate combined, and beta carotene. In addition to their free radical scavenging and antioxidant activity, proanthocyanidins have been reported to have antibacterial, antiviral, anticarcinogenic, antiinflammatory, and vasodilatory actions. Proanthocyanidins have also been shown to inhibit lipid peroxidation, platelet aggregation, capillary permeability and fragility, and to affect enzyme systems including phospholipase A2, cyclooxygenase, and lipoxygenase. It is unlikely these effects could lead to weight loss.

The effect of proanthocyanidins on obesity is not known; however, it was recently reported that GSE reduced lipolytic activity in 3T3-L1 adipocytes by inhibiting the fat mobilizing enzymes pancreatic lipase and lipoprotein lipase (Moreno D A, Ilic N, Poulev A, Brasaemle D L, Fried S K, Raskin I., Inhibitory effects of grape seed extract on lipases. Nutrition. 2003 October; 19(10):876-9, which is incorporated by reference herein in its entirety).

Embodiments of the present invention relate to the use of plant-derived substances to reduce the body fat in a mammalian subject. As used herein, the term “plant-derived” means a pure substance or a mixture of substances derived from whole or a part (root, stem, leaf, flower or fruit) of a flowering or non-flowering plant. Because these substances are endogenous to foods that are regularly consumed, they are less likely to have side-effects and, therefore, can be safely used on a long-term basis. As noted above, the determination of the amount of fat in the body depends on the generation of new fat cells (adipogenesis), the storage of fat inside those cells (lipogenesis), the breakdown of fat from those cells (lipolysis). It is submitted that the fat cell can compensate for any particular effect caused by any one compound. For example, if lipolysis is induced, the fat cell can compensate by increasing lipogenesis. Moreover, the body could generate more mature fat cells through adipogenesis in response to agents that inhibit lipogenesis and induce lipolysis. Therefore, combinations of compounds that attack multiple determinants of the fat depot should lead to more significant decreases than if the individual compounds given alone. (see, e.g., Duloo, A. G., Seydoux, J., Girardier, L., Chantre, P., & Vandermander, J., “Green tea and thermogenesis: interactions between catechin-polyphenols, caffeine and sympathetic activity, Int. J. Obes. Relat. Metab. Disord., vol. 24, pp. 252-58 (2000), which is incorporated by reference herein in its entirety, suggesting that a combination of caffeine and polyphenol is more effective than the sum of the individual components).

In certain embodiments, the invention comprises a composition for reducing the amount of fat in the body of a mammal, the composition comprising at least one plant-derived substance which inhibits adipogenesis in the body of the mammal and at least one plant-derived substance which promotes lipolysis in the body of the mammal. In certain embodiments, the invention comprises a composition for reducing the amount of fat in the body of a mammal, the composition comprising at least one plant-derived substance which inhibits adipogenesis in the body of the mammal, at least one plant-derived substance which promotes lipolysis in the body of the mammal, and at least one plant-derived substance which inhibits lipogenesis in the body of the mammal. The ideal amount of each compound to be used in the composition will be the smallest dose found to produce a reduction in body fat, and the ideal amount for each of the individual compounds may depend on the presences of and amounts of the other compounds in the composition.

In certain embodiments, capsaicin may be used in combination with other plant-derived substances to reduce the body fat in a mammalian subject. In one embodiment, an effective amount of a composition comprising capsaicin in combination with grape seed extract and/or genistein and/or caffeine may be administered to a subject to reduce the body fat of the subject. As used herein, the term “effective amount” means an amount of the composition that is sufficient to produce a reduction in the body fat of the subject. In one embodiment, the effective amount will be: capsaicin—2.5 mg; genistein—5 mg; Caffeine—100 mg; and grape seed extract—200 mg. In certain embodiments, capsaicin analogs and capsaicinoids may be used in the place of capsaicin.

In one embodiment, a composition comprising grape seed extract may be administered to a mammalian subject to increase the insulin sensitivity of the subject.

### EXAMPLE 1

### Capsaicin Inhibits Adipogenesis

It is reported here for the first time that capsaicin is a potent inhibitor of adipogenesis. The accepted model for the process of adipogenesis is the differentiation of mouse-derived 3T3-L1 fibroblasts into adipocytes after treatment with potent inducers such as dexamethasone, isobutyrylmethylxanthine, and insulin. In this model, the adipocytes are identified by the accumulation of lipid droplets in the cytoplasm. We tested the effect of the presence or absence of capsaicin on this process.

### Methods & Materials

NIH 3T3-L1 cells were obtained from American Type Culture Collection (Manassas, Va.). NIH 3T3-L1 cells are a mouse-derived fibroblast cell line that convert into adipocytes under certain conditions. This cell system is well known in the art and is a standard model for the study of adipocyte metabolism. The cells were cultured using well known methods in complete medium (Dulbecco Modified Eagle's Medium (DMEM) (4500 mg glucose/liter) containing 10% fetal bovine serum (FBS)). When the cells were 75% confluent, they were subcultured into multi-well culture plates and allowed to grow to confluency. The differentiation of the cells into adipocytes was initiated by switching to inducing medium (complete medium containing 10 uM dexamethasone, 0.5 mM isobutyrylmethylxanthine, and 10 ug/ml insulin). 1 ug/ml and 0.5 ug/ml doses of capsaicin were added to a number of the culture wells. After 48 hours, the inducing medium was replaced with maintenance medium 1 (complete medium containing insulin (10 ug/ml) with or without capsaicin). After 24 hours, the cells were switched to maintenance medium 2 (complete medium with or without capsaicin). The cultures were followed for up to 10 days for differentiation into adipocytes. The culture medium was replaced every 2 days with a medium of similar composition. The cells were photographed every 2 days to follow their progress. On day 10, the cells were stained with oil red-O (to stain for intracellular lipid) and photographed. For procedures, see Ramirez-Zacarias, J. L., Castro-Munozledo, F. & Kuri-Harcuch, W. Quantitation of adipose conversion and triglycerides by staining intracytoplasmic lipids with Oil red O. Histochemistry 97, 493-497 (1992), which is hereby incorporated by reference herein in its entirety.

### Results

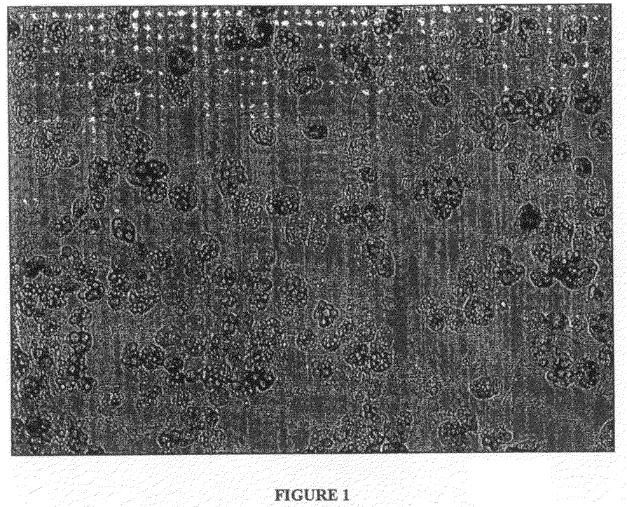

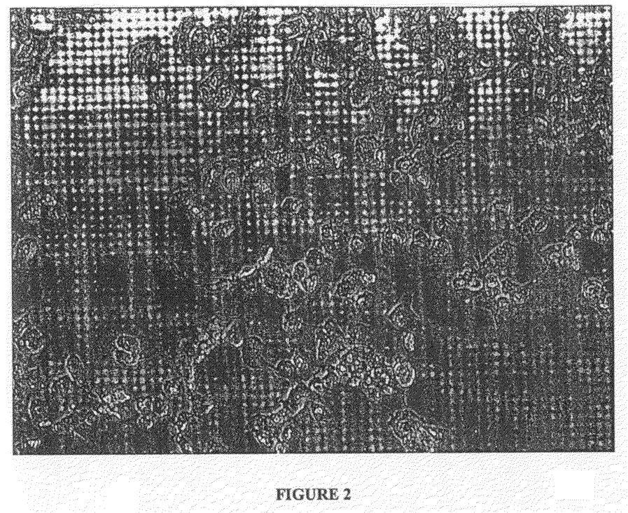

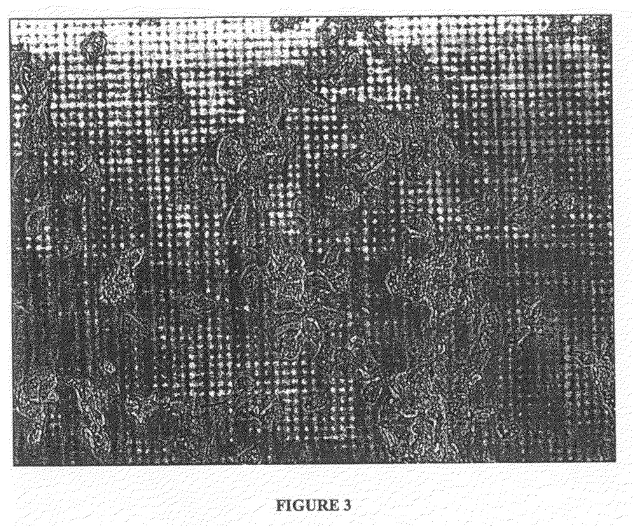

The results are shown in FIGS. 1-3. As shown in FIG. 1, 3T3-L1 cells exposed to the inducing medium followed by the maintenance media become loaded with intracellular lipid and assume the adipocyte phenotype. The presence of the intracellular lipid is illustrated by the dark color of the stain which can be seen in FIG. 1. Cells exposed to 0.5 ug/ml (FIG. 2) and 1 ug/ml (FIG. 3) capsaicin had a significant decrease in lipid accumulation and did not become adipocytes, even after 10 days. The lack of lipid accumulation is illustrated in FIGS. 2 and 3 by the lack of dark stain; it can also be seen that the cells in these figures have not taken on the round shape of adipocyte cells (for comparison see FIG. 1).

### EXAMPLE 2

### Grape Seed Extract Stimulates Lipolysis

### Breakdown of Fat

The data presented below demonstrate for the first time another biologic activity of GSE that will be beneficial in reducing body fat: the ability to promote lipolysis.

### Methods & Materials

NIH 3T3-L1 cells were obtained from American Type Culture Collection (Manassas, Va.). The cells were cultured in Dulbecco Modified Eagle's Medium (DMEM) (4500 mg glucose/liter) containing 10% fetal bovine serum (FBS). When the cells were 75% confluent, they were subcultured into multi-well culture plates and allowed to grow to confluency. The differentiation of the cells into adipocytes was initiated by the addition of 10 uM dexamethasone, 0.5 mM isobutyrylmethylxanthine, and 10 ug/ml insulin to the culture medium for 2 days, followed by the cultivation of the cells in culture medium without dexamethasone, isobutyrylmethylxanthine, and insulin for an additional 3 or more days. See Frost S. C., Lane M. D., Evidence for the involvement of vicinal sulfhydryl groups in insulin-activated hexose transport by 3T3-L1 adipocytes. J Biol. Chem. 1985 Mar. 10; 260(5):2646-52, which is incorporated herein by reference in its entirety.

Mature 3T3-L1 adipocytes in 24-well culture plates were incubated in either complete medium (DMEM and 10% FBS), or complete medium containing 5 ul of grape seed extract (500 μg/ml dissolved in 50% ethanol), or an equivalent volume of 50% ethanol in a CO₂ incubator. After 2 hours, the medium was removed from each well and the amount of glycerol released into the medium was measured by radiometric assay of glycerol using the method described in D. C. Bradley, H. R. Kaslow, Radiometric assays for glycerol, glucose and glycogen, Anal. Biochem. 180 (1989) 11-16, which is hereby incorporated by reference herein in its entirety.

### Results

The data are presented in TABLE 2 below. Grape seed extract (500 ug/ml) caused a 58% increase in adipocyte lipolysis over baseline and the ethanol-treated control cells.

TABLE 2

| | Lipolysis | |
|---|---|---|
| Additions | (CPM × 10⁶/well) | p-Value |
| None | 995 ± 138 | N/A |
| Ethanol | 981 ± 178 | N/A |
| Grape seed extract (500 μg/ml | 1579 ± 33 | 0.0046 |

### EXAMPLE 3

### Effect of a Combination of Grape Seed Extract, Capsaicin, Genistein, and Caffeine on Body Weight and Adipose Tissue Fat Content

Compositions consisting of grape seed extract, capsaicin, genistein, and Caffeine were examined to determine their effect on body weight and adiposity of obese Zucker rats fed a high fat diet.

### Methods & Materials

20 Adult male obese Zucker rats were fed a high fat base diet (50% Fat, 30% protein, and 20% carbohydrate calories). Experimental groups were given treatments with two diets, Diet A or Diet B, each of which comprised the ingredients listed in TABLE 3 below.

TABLE 3

| | Diet A | Diet B |
|---|---|---|
| | (per kg of high fat | (per kg of high fat |
| | base diet) | base diet) |
| Grape seed extract | 20 g | 10 g |
| Genistein | 600 mg | 300 mg |
| Capsaicin | 600 mg | 300 mg |
| Caffeine | 12 g | 6 g |

The feeding procedures were as follows: twenty rats were grouped into 10 pairs based on body weight. Five pairs were assigned to each diet group. The daily food intakes of control (treatment-free diet) and treated (treatment diet) animals in each pair were recorded. On the following day the control rat in each pair received the same weight of treatment-free diet as consumed the previous day by its partner under treatment. Therefore, both animals in the pair received the same number of calories of food over the duration of the experiment.

### Results

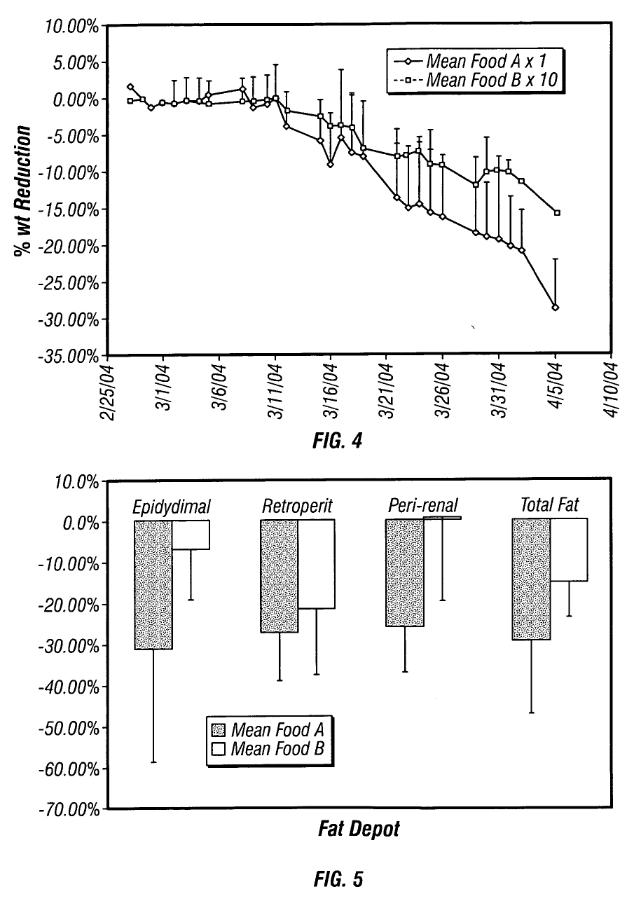

The data presented in FIG. 4 show that there was no change in the body weight of animals on Diet A or Diet B for the first two weeks. However, after the first two weeks, there appeared a steady decline in the body weight of animals in both groups. Towards week 5 of treatment, the Diet A group had lost about 30% body weight and the Diet B group about 16%. These data suggest a dose dependant response in the pharmacological actions of the composition.

At week 5, when the experiments were terminated, the animals were sacrificed by decapitation and the size of 3 different fat depots (epidydimal, retroperitoneal, and peri-renal) was measured. The data presented in FIG. 5 show that there was 25% to 30% loss in all fat depots in the animals receiving Diet A. The animals receiving Diet B lost almost the same level of retroperitoneal fat, less of epidydimal fat, and none of peri-renal fat as compared with those receiving Diet A.

Treatment with a mixture of GSE, Genistein, Capsaicin, and Caffeine causes dose-dependent weight loss. Because these animals were pair-fed, weight loss observed may have resulted from a combination of increased energy expenditure/loss and decreased accretion of fat in the body. Concomitant with the decrease in body weight, we observed a decrease in body fat in all depots on diet A. The retroperitoneal fat depot appears to be very sensitive to the treatment. It is also known that it is the retroperitoneal depot that has been associated with many of the ill effects of increased adiposity. In this sense, a decrease in this fat depot may carry many health benefits.

Experience of the inventors has shown that if an agent is effective in a rat model at 1 mg/Kg, an average (70-80 Kg) human will need about 1 mg/day. Therefore, based on rat data, it is submitted that the following dosage (plant-derived compositions per pill, administered 3 times a day with meal) could be effective for an average human: capsaicin—2.5 mg; genistein—5 mg; Caffeine—100 mg; and GSE—200 mg.

### EXAMPLE 4

### A Combination of Grape Seed Extract, Capsaicin, Genistein, and Caffeine Causes Decreased TNF-Alpha and Increased Adiponectin in Obese Zucker Rats

TNF-alpha is elevated in obese individuals and is involved in the development of insulin resistance (Moller, D. E., Kaufman, K. D., Metabolic Syndrome: A Clinical and Molecular Perspective. Annu. Rev. Med., 2004 Aug. 11, which is hereby incorporated by reference herein in its entirety). Adiponectin, an adipohormone, improves insulin sensitivity in tissues and is regulated by TNF-alpha (ibid.). To investigate whether a combination of grape seed extract, capsaicin, genistein, and caffeine altered these parameters, TNF-alpha (by ELISA, Biosource, Camarillo, Calif.) and adiponectin (by ELISA, B-Bridge International, Sunnyvale, Calif.) were assayed using methods well known in the art using blood obtained at sacrifice from animals fed Diet B as discussed above in Example 3 (Ed Harlow and David Lane (1988) Antibodies A Laboratory Manual pp 553-612 ColdSpring Harbor Laboratory). Despite variation in levels (ranging from 2.40 to 83.45 pg/ml serum), animals fed Diet B on average had less serum TNF-α over that of control pair-fed animals (15.12 pg/ml+/−8.130 vs 29.24 pg/ml+/−13.97; mean+/−SEM pg TNF-alpha/ml serum). In addition, animals treated with Diet B had 40% more adiponectin (5.43 ug/ml+/−1.7 ug/ml vs 3.84+/−0.26 ug/ml serum; mean+/−SD; p=0.059).

### EXAMPLE 5

### Rat Adipocytes from Combination Fed Rat have Enhanced Response to Stimulators of Lipolysis

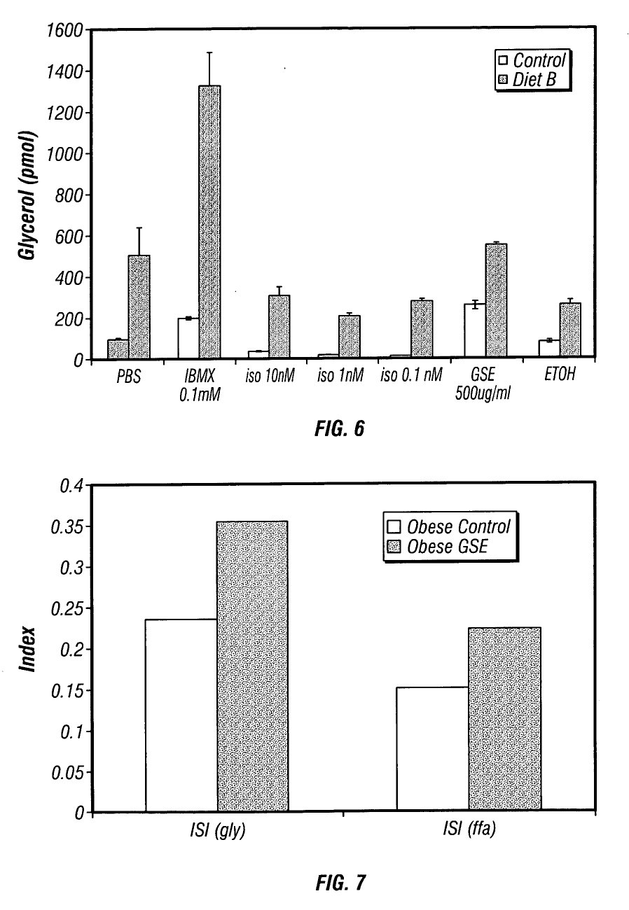

Obese Zucker rats are known to have reduced response to stimulators of lipolysis, including the phosphodiesterase inhibitor IBMX and the beta-adrenergic agonist isoproterenol (Gema Frühbeck and Javier Gomez-Ambrosi (2002). Depot-specific differences in the lipolytic effect of leptin on isolated white adipocytes. Signature: Med Sci Monit., 2002; 8(2):BR47-55, which is hereby incorporated by reference herein in its entirety. We isolated primary adipocytes from a rat treated with Diet B as discussed above in Example 3 and from its control pair-fed rat and tested both stimuli in an in vitro lipolysis assay. Adipocytes were prepared from the retroperitoneal fat depots and lipolysis assays were performed as described in Figueroa, J. E. 2nd. Vijayagopal, P., Prasad, C. (2002) Azaftig stimulates in vitro lipolysis by rodent and human adipocytes. Biochem Biophys Res Comm. 293:847-9, which is hereby incorporated by reference herein in its entirety. Adipocytes were exposed to PBS; 0.1 mM IBMX; 10 uM isoproterenol; 1 uM isoproterenol; 0.1 uM isoproterenol; 500 ug/ml GSE; or ETOH. The data are shown in FIG. 6. The adipocytes from the rat treated with Diet B showed increased baseline lipolysis compared with control. Uniformly, the adipocytes from the Diet B-treated rat were more responsive than the control adipocytes. These data demonstrate that treatment with a composition comprising grape seed extract, capsaicin, genistein, and Caffeine enhances lipolysis both at baseline and from other stimuli.

### EXAMPLE 6

### Treatment of Obese Zucker Rats with GSE Improves Insulin Sensitivity

Diabetes results from insulin resistance (decreased insulin sensitivity means increased insulin resistance and vice versa). A decrease in the ability of insulin to control normal blood glucose means increased insulin resistance. In the majority of people with type 2 diabetes, the body's cells ignore the body's insulin. Insulin is a hormone made by the pancreas that allows the body's cells to use sugar by taking the sugar from the blood into the cells. If sugar does not enter the cells, it can build up in the blood, which has unhealthy side effects.

Insulin resistance can lead to high blood sugar. When a person eats, food enters the digestive system where it's broken down into basic elements, including glucose, an important sugar which is the body's primary energy source. Glucose is commonly known as “blood sugar.”

Following digestion, insulin and blood sugar enter the bloodstream. In people without insulin resistance, insulin allows sugar to enter the body's muscle, fat and liver cells easily and efficiently. For people who have insulin resistance (an estimated 90% of people with type 2 diabetes), it is more difficult for blood sugar to enter muscle, fat and liver cells. This difficulty can cause high levels of glucose to accumulate in the blood, which can lead to many short-term and long-term health problems, including eye problems, blindness, kidney damage, nerve damage, lower-limb amputation and heart disease. Because the cells are energy-starved, the pancreas may begin to work harder to produce more insulin in an effort to provide more glucose for cells. This extra effort can cause the pancreas to lose its ability to produce sufficient amounts of insulin. See Jerrold M. Olefsky (1989) Pathogenesis of Type II diabetes, In: Endocrinology, Vol. 2 (Edited by L. J. DeGroot), pp. 1369-1388, which is hereby incorporated by reference herein in its entirety.

The ability of GSE to alter insulin resistance in obese Zucker rats was examined after feeding a 50% fat diet containing 2%. Animals were fed using the feeding procedures discussed above in EXAMPLE 3. Using methods well known in the art, sera were assayed for glucose (Glucose oxidase assay—Pointe Scientific, Lincoln Park, Mich.), free fatty acids (ffa; NEFA calorimetric assay kit—Wako Chemicals, Germany), and insulin (Ultrasensitive rat insulin ELISA kit—Crystal Chem, Inc., Downers Grove, Ill.). These data were analyzed to determine the insulin sensitivity indices (ISI) of individual animals for glycemia [ISI(gly)] and plasma FFA [ISI(ffa)] using the following formulas:

ISI(gly)=2/[(fasting plasma insulin in uM)×(fasting blood glucose in mM)+1]

ISI(ffa)=2/[(fasting plasma insulin in uM)×(fasting plasma FFA in mM)+1]

(see Belfiore, F., Iannelo, S., and Volpicelli, G., Insulin sensitivity indices calculated from basal and OGTT-indiced insulin, glucose, and FFA levels, Mol Genet Metab 63:134-141, 1998, which is hereby incorporated by reference herein in its entirety). The mean ISI(gly) and ISI(ffa) are shown in FIG. 7. The GSE-treated animals had improved insulin sensitivity (as indicated by the higher ISI index) than control animals. These data, in conjunction with the increased adiponectin (see Example 4 above) seen with GSE treatment, demonstrate that GSE works to reverse obesity-related insulin resistance.

The descriptions of the foregoing embodiments of the invention have been presented for purpose of illustration and description. They are not intended to be exhaustive or to limit the invention to the precise forms disclosed, and obviously many modifications and variations are possible in light of the above teaching. The embodiments were chosen and described in order to best explain the principles of the invention to thereby enable others skilled in the art to utilize the invention in various embodiments and with various modifications as are suited to the particular use contemplated. It is intended that the scope of the invention be defined by the claims appended hereto.

We claim:

1. A composition for reducing the amount of fat in the body of a mammal, said composition comprising:
a. at least one plant-derived substance which inhibits adipogenesis in the body of said mammal; and
b. at least one plant-derived substance which promotes lipolysis in the body of said mammal.

2. The composition of claim 1, wherein said at least one plant-derived substance which inhibits adipogenesis comprises capsaicin.

3. The composition of claim 1, wherein said at least one plant-derived substance which inhibits adipogenesis comprises a capsaicin analog.

4. The composition of claim 3, wherein said capsaicin analog comprises at least one C14 to C20 alkyl side chain.

5. The composition of claim 4, wherein said capsaicin analog is capsiate or dihydrocapsiate.

6. The composition of claim 1, wherein said at least one plant-derived substance which promotes lipolysis comprises grape seed extract.

7. The composition of claim 1, further comprising at least one plant-derived substance which inhibits lipogenesis in the body of said mammal.

8. The composition of claim 7, wherein said at least one plant-derived substance which inhibits lipogenesis comprises genistein.

9. A composition for reducing the amount of fat in the body of a mammal, said composition comprising at least two of the following:
a. grape seed extract;
b. capsaicin;
c. genistein; and
d. caffeine.

10. The composition of claim 9, wherein said composition comprises capsaicin and grape seed extract.

11. The composition of claim 10, wherein said composition further comprises genistein and caffeine.

12. The composition of claim 11, wherein the ratio by weight of grape seed extract to genistein and capsaicin is about 1:30, the ratio by weight of grape seed extract to caffeine is about 1:0.6, the ratio by weight of genistein to capsaicin is about 1:1, the ratio by weight of genistein to caffeine is about 1:0.02, and the ratio by weight of capsaicin to caffeine is about 1:0.02.

13. A composition for reducing the amount of fat in the body of a mammal, said composition comprising at least two of the following:
a. grape seed extract;
b. a capsaicin analog
c. genistein; and
d. caffeine.

14. The composition of claim 13, wherein said composition comprises a capsaicin analog and grape seed extract.

15. The composition of claim 14, wherein said capsaicin analog comprises at least one C14 to C20 alkyl side chain.

16. The composition of claim 15, wherein said capsaicin analog is capsiate or dihydrocapsiate.

17. The composition of claim 14, wherein said composition further comprises genistein and caffeine.

18. A method for reducing the amount of fat in the body of a mammalian subject by administering an effective amount of a composition to said subject, said composition comprising at least two of the following:
a. grape seed extract;
b. capsaicin;
c. genistein; and
d. caffeine.

19. The method of claim 18, wherein said composition comprises capsaicin and grape seed extract.

20. The method of claim 19, wherein said composition further comprises genistein and caffeine.

21. A method for reducing the amount of fat in the body of a mammalian subject by administering an effective amount of a composition to said subject, said composition comprising at least two of the following:
a. grape seed extract;
b. a capsaicin analog;
c. genistein; and
d. caffeine.

22. The method of claim 21, wherein said composition comprises a capsaicin analog and grape seed extract.

23. The method of claim 21, wherein said capsaicin analog comprises at least one C14 to C20 alkyl side chain.

24. The method of claim 23, wherein said capsaicin analog is capsiate or dihydrocapsiate.

25. The method of claim 22, wherein said composition further comprises genistein and caffeine.

26. A method for increasing insulin sensitivity in a mammalian subject by administering an effective amount of a composition to said subject, said composition comprising grape seed extract.

27. The method of claim 26, wherein said mammalian subject is obese.

28. The method of claim 26, wherein said composition further comprises capsaicin and grape seed extract.

29. The method of claim 27, wherein said composition further comprises genistein and caffeine.
